(19) United States
(12) Patent Application Publication
(10) Pub. No.: US 2019/0374171 A1
(43) Pub. Date: Dec. 12, 2019

(54) Method of Analyzing Psychological Signals and Related Analyzing Device

(71) Applicant: Wistron Corporation, New Taipei City (TW)

(72) Inventor: Fang-Ming Wu, New Taipei City (TW)

(21) Appl. No.: 16/105,994

(22) Filed: Aug. 21, 2018

(30) Foreign Application Priority Data
Jun. 11, 2018 (TW) 107120073

(51) Int. Cl. A61B 5/00; A61B 5/0205

(52) U.S. Cl. CPC A61B 5/7278; A61B 5/024; A61B 5/7225; A61B 5/0205

(57) ABSTRACT

A method of analyzing a psychological signal includes receiving a reflective signal corresponding to the psychological signal reflected by a user; performing summation to the reflective signal and a probe signal having a first frequency, to generate a summation signal; and performing a subspace analysis of a first dimension to the summation signal, to generate an analytic result, to determine a frequency of the psychological signal.

### BACKGROUND OF THE INVENTION

### 1. Field of the Invention

The present invention relates to a method of analyzing psychological signals and related analyzing device, and more particularly, to a method and related analyzing device using a subspace analysis to analyze the psychological signals.

### 2. Description of the Prior Art

The conventional ways of obtaining a psychological signal from a user may be categorized into a contact way and a non-contact way. In general, the contact way obtains the psychological signal through directly contacting the user body, which may obtain the psychological signal more accurately and with better reliability. However, under such a circumstance that a sensing device is required to be worn by the user for long time, the sensing device often falls off from the user body, or the user may not use the sensing device of the contact way under some certain scenarios. As such, the psychological signal of the user is not able to be obtained through the sensing device of the contact way, and has to be obtained through the sensing device of the non-contact way.

The conventional non-contact way to obtain the psychological signal may though meet requirements of the user, or overcome the scenarios that the user cannot wear the wearable sensing device. However, the psychological signal obtained through the non-contact way is easily affected by factors of the environmental noise, the signal intensity, the blocked medium, etc., which degrade the accuracy or the reliability. Therefore, to analyze the psychological signal obtained through the non-contact way, the prior art usually utilizes an analyzing method such as the short-time Fourier transform (STFT), the wavelet transform, the empirical mode decomposition, etc., for determining the psychological frequency from the signal. Therefore, the prior art takes much computation time with no guarantee of obtaining the correct psychological signal, which leads to degradations of the analyzing result on the accuracy and the reliability, and deteriorates when the signal intensity becomes weaker.

Therefore, how to fast and accurately analyze the psychological signal for obtaining the psychological frequency thereof is a significant objective in the field.

### SUMMARY OF THE INVENTION

It is therefore a primary objective of the present invention to provide a method of analyzing psychological signals and a related analyzing device capable of accurately analyzing a psychological signal, to improve over the prior art.

The present invention discloses a method of analyzing a psychological signal, comprising receiving a reflective signal corresponding to the psychological signal reflected by a user; performing summation to the reflective signal and a probe signal having a first frequency, to generate a summation signal; and performing a subspace analysis of a first dimension to the summation signal, to generate an analytic result, to determine a frequency of the psychological signal.

The present invention further discloses an analyzing device, for analyzing a psychological signal. The analyzing device comprises a receiver, configured to receive a reflective signal corresponding to the psychological signal reflected by a user; a probing unit, configured to perform summation to the reflective signal and a probe signal having a first frequency, to generate a summation signal; and an analyzing module, configured to perform a subspace analysis of a first dimension to the summation signal, to generate an analytic result, to determine a frequency of the psychological signal.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of an analyzing device according to an embodiment of the present invention.

FIG. 2A is a schematic diagram of a process according to an embodiment of the present invention.

FIG. 2B is a schematic diagram of another process according to an embodiment of the present invention.

FIG. 3A illustrates a schematic diagram of a reflective signal according to an embodiment of the present invention.

FIG. 3B illustrates a schematic diagram of a summation signal according to an embodiment of the present invention.

FIG. 4A illustrates a schematic diagram of a noise subspace pseudospectrum according to an embodiment of the present invention.

FIG. 4B illustrates a schematic diagram of a signal subspace pseudospectrum according to an embodiment of the present invention.

FIG. 5A illustrates a schematic diagram of another noise subspace pseudospectrum according to an embodiment of the present invention.

FIG. 5B illustrates a schematic diagram of another signal subspace pseudospectrum according to an embodiment of the present invention.

FIG. 6 is a schematic diagram of another analyzing device according to an embodiment of the present invention.

### DETAILED DESCRIPTION

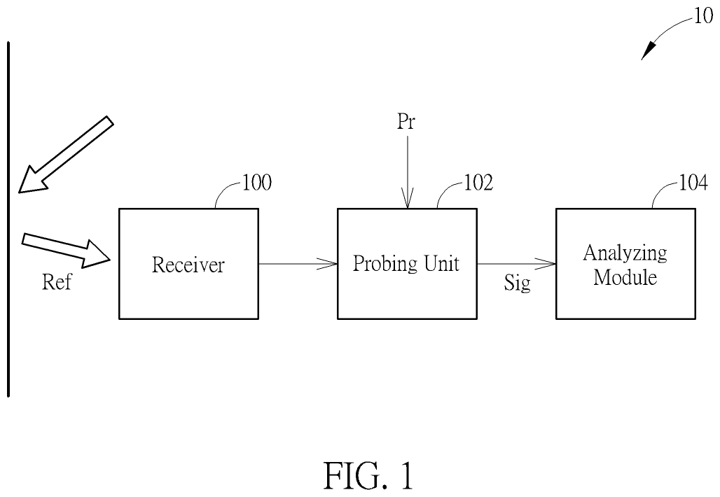

Please refer to FIG. 1, which is a schematic diagram of an analyzing device 10 according to an embodiment of the present invention. The analyzing device 10 may receive a reflective signal Ref reflected by a user's body, and analyze a psychological frequency of the user carried by the reflective signal Ref. The analyzing device 10 includes a receiver 100, a probing unit 102 and an analyzing module 104. The receiver 100 is configured to receive the reflective signal Ref reflected by the user. Since the reflective signal Ref is reflected from the user, a psychological signal corresponding to the psychological frequency of the user may be carried by the reflective signal Ref. The probing unit 102 is coupled to the receiver 100, configured to perform a summation of the reflective signal Ref and a probe signal Pr having a first frequency Freq1, so as to generate a summation signal Sig. The analyzing module 104 is coupled to the probing unit 102, configured to perform a subspace analysis to the summation signal Sig, for generating an analytic result to determine a frequency of the psychological signal.

In the embodiment, the receiver 100 may be a wireless receiver, configured to receive the psychological signal with the psychological frequency reflected by the user, wherein the reflective signal Ref, received by the receiver 100, may be generated by a transmitter, which is not illustrated in FIG. 1, for emitting an incident signal to the user. Thus, the type and the specification of the receiver 100 may be preferably selected to meet different application requirements and design concepts demanded by the transmitter and the user, for example, a frequency range of the psychological signal intended to be measured, a specification of the transmitter, a frequency range of the incident signal, a signal intensity, a penetration ability of the wireless signal, etc. Therefore, the receiver 100 may correctly receive the reflective signal Ref which is generated by the transmitter and emitted to the user, such that the analyzing module 104 may perform corresponding analysis. Moreover, the probing unit 102 may be a summation circuit of amplitude, configured to perform the summation of an amplitude of the reflective signal Ref and an amplitude of the probe signal Pr, for generating the summation signal Sig. The analyzing module 104 may be a microprocessor (MCU) or an application-specific integrated circuit (ASIC), configured to analyze the summation signal Sig according to the subspace analysis. In addition, the embodiment aims at illustrating the analyzing device 10, but not limiting the scope of the present invention. For example, the probing unit 102 and the analyzing module 104 may be integrated into a single chip, an MCU, an ASIC or a processor through a system on chip (SoC) method, which are within the scope of the present invention.

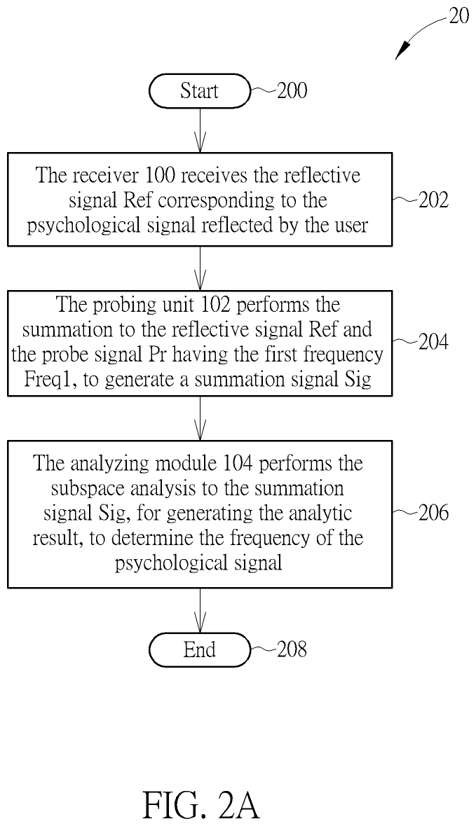

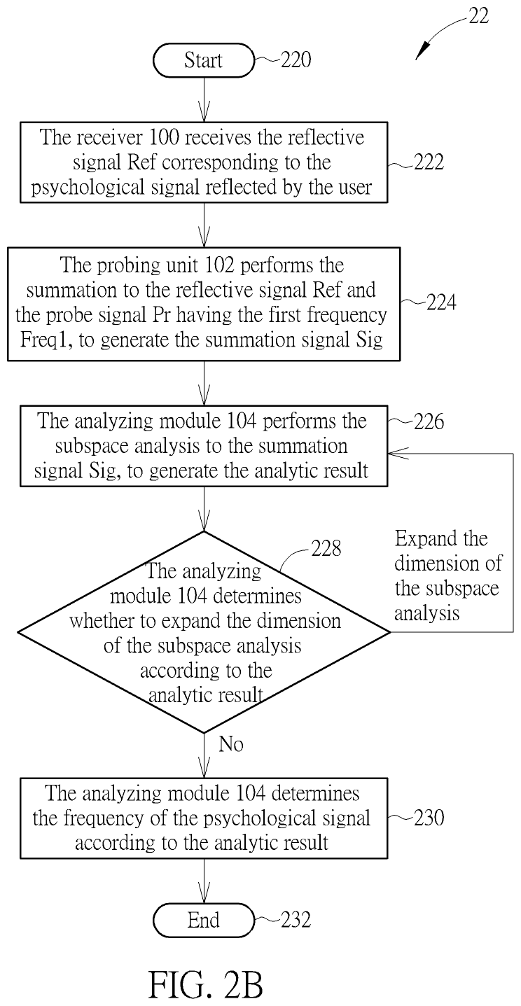

The operations of the analyzing device 10 may be summarized as a process 20, as shown in FIG. 2A. The process 20 comprises the following steps:

Step 200: Start.

Step 202: The receiver 100 receives the reflective signal Ref corresponding to the psychological signal reflected by the user.

Step 204: The probing unit 102 performs the summation to the reflective signal Ref and the probe signal Pr having the first frequency Freq1, to generate a summation signal Sig.

Step 206: The analyzing module 104 performs the subspace analysis to the summation signal Sig, for generating the analytic result, to determine the frequency of the psychological signal.

Step 208: End.

According to the process 20, in Step 202, the receiver 100 receives the reflective signal Ref corresponding to the psychological signal reflected by the user's body. In Step 204, the probing unit 102 estimates a power of the reflective signal Ref for determining a power of the probe signal Pr, and performing the summation to the probe signal Pr and the reflective signal Ref to generate the summation signal Sig. Furthermore, an intensity of the reflective signal Ref received by the probing unit 102 varies under different circumstances (e.g., different distances between the receiver 100 and the user's body, or different intensities of the incident signal). Thus, the probing unit 102 estimates the power of the reflective signal Ref in advance, such that the analyzing module 104 may preferably determine the power and the amplitude of the reflective signal Ref according to the power estimated by the probing unit 102. Under such a circumstance, the analyzing device 10 may determine the intensity of the probe signal Pr (e.g., the amplitude of the probe signal Pr may be set to 1/12 or 1/20 times of the intensity of the reflective signal Ref) according to the intensity and/or the amplitude of the reflective signal Ref.

Next, in Step 206, the analyzing module 104 may initially perform the subspace analysis of an N dimensions to the summation signal Sig, for generating the analytic result to determine the frequency of the psychological signal, wherein the analyzing module 104 may estimate a composition in frequency of the summation signal Sig according to a multiple signal classification (MUSIC) algorithm, to determine the frequency of the psychological signal.

More specifically, the analyzing module 104 may transform the summation signal Sig to a summation matrix S(t) of dimension N according to the dimension of the subspace analysis in advance, and perform a covariance matrix operation to the summation matrix, S(t) for obtaining the covariance matrix R according to the following eq. (1). As such, a homology of the summation signal Sig may be decreased, and thus, increasing a measurement accuracy.

R=E[SS^{H}] (1)

Then, the analyzing module 104 performs a singular value decomposition (SVD) to the covariance matrix R, for obtaining an eigenvalue matrix Λ and an eigenvector matrix V according to the following eq. (2).

R=VΛV^{H} (2)

At last, a signal subspace and a noise subspace may be decomposed by the analyzing module 104 from the eigenvectors of the eigenvector matrix V according to the following eq. (3), such that the noise subspace is orthogonal to the signal subspace composed by the frequency vectors.

VΛV^{H}=\hat{V}_{S}\hat{Λ}_{S}\hat{V}_{S}^{H}+\hat{V}_{N}\hat{Λ}_{N}\hat{V}_{N}^{H} (3)

It is noted that the \hat{Λ}_{S} is denoted as the eigenvector of the signal subspace; the \hat{Λ}_{N} is denoted as the eigenvector of the noise subspace; the \hat{V}_{S} is denoted as the eigenvector of the signal subspace; the \hat{V}_{N} is denoted as the eigenvector of the noise subspace. Therefore, the signal subspace and the noise subspace may be decomposed by the analyzing module 104 according to eq. (3), such that the analyzing module 104 may determine the frequency of the psychological signal according to the signal subspace.

The detailed operations of the analyzing device 10 mentioned above may be summarized as another process 22, as shown in FIG. 2B. The process 22 comprises the following steps:

Step 220: Start.

Step 222: The receiver 100 receives the reflective signal Ref corresponding to the psychological signal reflected by the user.

Step 224: The probing unit 102 performs the summation to the reflective signal Ref and the probe signal Pr having the first frequency Freq1, to generate the summation signal Sig.

Step 226: The analyzing module 104 performs the subspace analysis to the summation signal Sig, to generate the analytic result.

Step 228: The analyzing module 104 determines whether to expand the dimension of the subspace analysis according to the analytic result. If yes, expand the dimension of the subspace analysis, and return to Step 226; if not, perform Step 230.

Step 230: The analyzing module 104 determines the frequency of the psychological signal according to the analytic result.

Step 232: End.

Steps 220-224 are similar to Steps 200-204, which are not narrated herein. Notably, in Step 226, the analyzing module 104 may generate a noise subspace pseudospectrum and a signal subspace pseudospectrum according to eq. (3), and determine the frequency of the psychological signal according to the intensity at the first frequency Freq1 in the noise subspace pseudospectrum. When the intensity of the probe signal Pr in the noise subspace pseudospectrum is greater than a predetermined intensity A1, it stands for that the dimension of the summation matrix S(t) is fine enough to extract the noise from the signal. The result of determining the probe signal Pr performed by the analyzing module 104 is not affected by the noise, and the analyzing module 104 may perform Step 230 for analyzing the signal subspace pseudospectrum, to obtain the frequency of the psychological signal. On the contrary, when the intensity of the probe signal Pr at the first frequency Freq1 in the noise subspace pseudospectrum is smaller than or equal to the predetermined intensity A1, it stands for that the dimension of the summation matrix S(t) is not fine enough to extract the noise from the signal. The result of determining the probe signal Pr performed by the analyzing module 104 is affected by the noise, such that the analyzing module 104 may expand the dimension of the signal subspace and the noise subspace (i.e. expand the dimension of the summation matrix S(t) and the subspace analysis). Then, the analyzing module 104 repeatedly performs Step 226 with the summation matrix S(t) of expanded and finer dimension, until the analyzing module 104 determines that the intensity of the probe signal Pr at the first frequency Freq1 in the noise subspace pseudospectrum is greater than the predetermined intensity A1, which represents that the dimension of the summation matrix S(t) is fine enough to extract the noise from the signal. The analyzing module 104 may obtain the frequency of the psychological signal according to the signal subspace pseudospectrum.

In brief, the analyzing device 10 of the present embodiment may receive the reflective signal reflected by the user, to determine the frequency of the psychological signal of the user. When the received reflective signal Ref is affected by the noise, the analyzing device 10 of the embodiment may accurately analyze the frequency of the psychological signal of the user according to the MUSIC algorithm without being affected by the environmental noise.

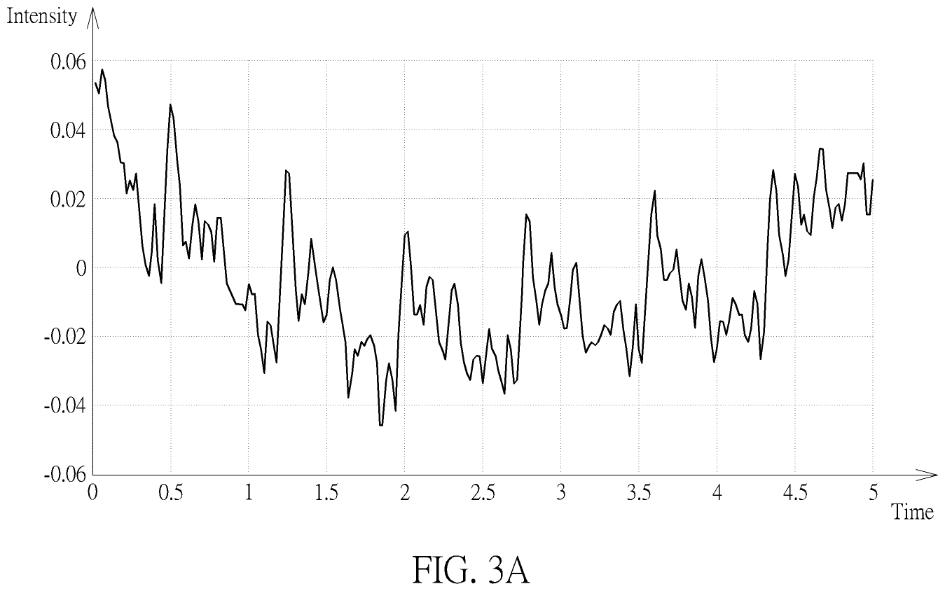

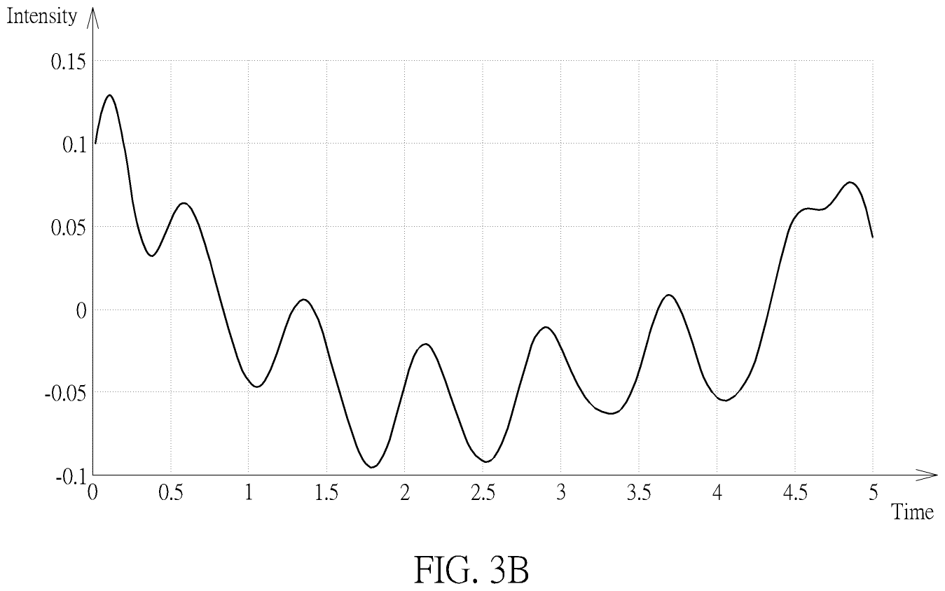

Please refer to FIGS. 3A-3B, wherein FIG. 3A illustrates a schematic diagram of the reflective signal Ref according to an embodiment of the present invention, and FIG. 3B illustrates a schematic diagram of the summation signal Sig according to an embodiment of the present invention. As shown in FIG. 3A, the receiver 100 receives the reflective signal Ref reflected by the user, and variations corresponding to the psychological signal of the user (e.g. frequency, amplitude or phase) may be carried by the reflective signal Ref. However, the reflective signal Ref having the psychological signal is weaker, which is prone to be affected by the environmental noise, and thus, harder to be obtained. Therefore, as shown in FIG. 3B, the probing unit 102 may perform the summation to the reflective signal Ref and the probe signal Pr, for generating the summation signal Sig, to facilitate the analyzing module 104 for analyzing the summation signal Sig and determining the frequency of the psychological signal.

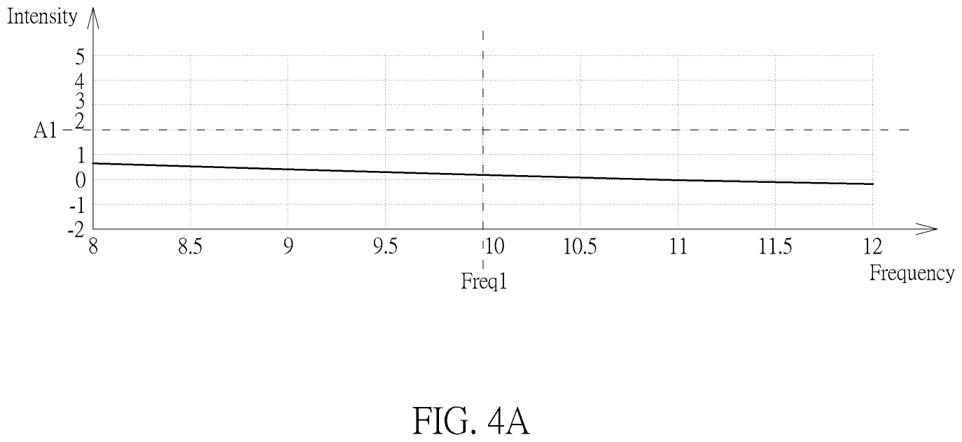

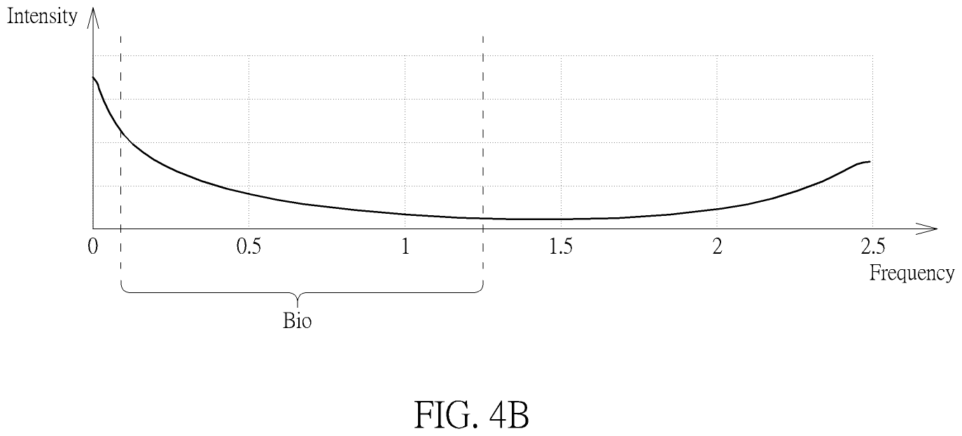

Next, please refer to FIGS. 4A, 4B, wherein FIG. 4A illustrates a schematic diagram of a noise subspace pseudospectrum generated by the analyzing module 104, and FIG. 4B illustrates a schematic diagram of a signal subspace pseudospectrum generated by the analyzing module 104. The analyzing module 104 may analyze the summation signal Sig according to the MUSIC algorithm, such that the noise subspace pseudospectrum, as shown in FIG. 4, may be generated. The intensity of the probe signal Pr at the first frequency Freq1 in the noise subspace pseudospectrum is smaller than the predetermined intensity A1, and thus, the analyzing module 104 determines that the dimension of the subspace analysis is not fine enough to obtain the psychological signal of the reflective signal Ref according to the noise subspace pseudospectrum. Therefore, the dimension of the subspace analysis is increased and Step 206 is repeated. The analyzing module 104 may analyze the summation signal Sig according to the MUSIC algorithm, such that the signal subspace pseudospectrum, as shown in FIG. 4B, may be generated. Under such a circumstance that the intensity of the probe signal Pr in the noise subspace pseudospectrum is smaller than the predetermined intensity A1, no wave crest of the psychological frequency is existed inside the frequency range Bio in the signal subspace pseudospectrum, and the analyzing module 104 may not determine the frequency of the psychological signal.

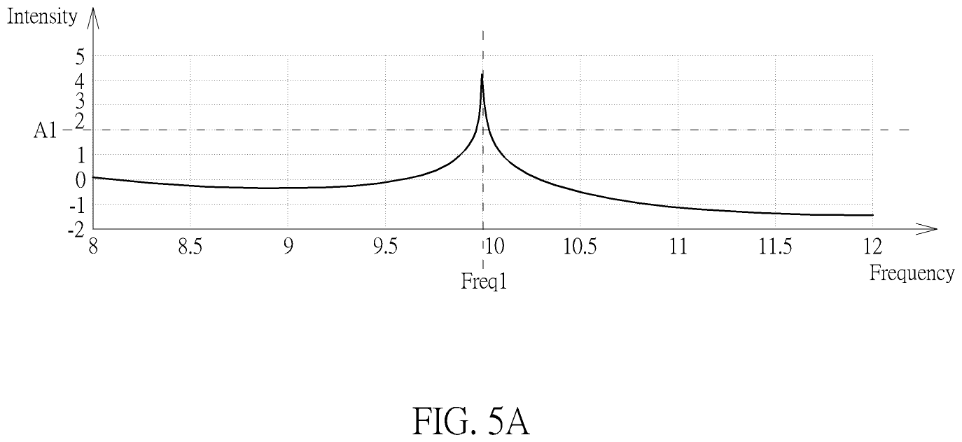

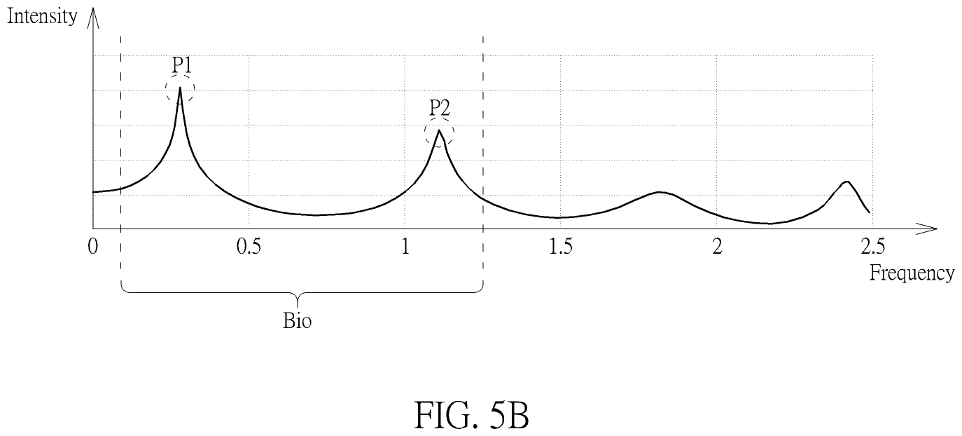

After at least one iteration of Step 206 are performed by the analyzing device 10, please refer to FIGS. 5A-5B, wherein FIG. 5A illustrates a schematic diagram of another noise subspace pseudospectrum generated by the analyzing module 104, and FIG. 5B illustrates a schematic diagram of another signal subspace pseudospectrum generated by the analyzing module 104. As shown in FIG. 5A, the intensity of the probe signal Pr at the first frequency Freq1 in the noise subspace pseudospectrum is greater than the predetermined intensity A1. Under such a circumstance, as shown in FIG. 5B, wave crests P1, P2 of the psychological frequency are existed inside the frequency range Bio in the signal subspace pseudospectrum, such that the analyzing module 104 may determine the frequency of the psychological signal. Therefore, when the intensity of the probe signal Pr is greater than the predetermined intensity A1, the analyzing module 104 may determine the frequency of the psychological signal according to where the frequency of the wave crests are located in the signal subspace pseudospectrum.

Notably, the embodiments stated in the above are utilized for illustrating the concept of the present invention. Those skilled in the art may make modifications and alterations accordingly, and not limited herein. For example, the receiver disclosed in an embodiment of the present invention, utilized for receiving the psychological signal of certain frequency bands, comprising the psychological frequency, may be the wireless receiver. In another embodiment, the receiver of the present invention may be a light sensor, and the reflective signal Ref recited by the receiver may be an amplitude signal of light or a brightness signal. Under such a circumstance, although the light senor is utilized for receiving the amplitude signal of light, the light sensor may continuously record the amplitude signal of light reflected by the user, and transform the amplitude signal of light with a certain length of time from a time domain to a frequency domain, for analyzing the frequency of the psychological signal. Therefore, the analyzing device of the present invention may be applied to different receivers according to different user requirements and design concepts, further increasing hardware compatibility of the analyzing device.

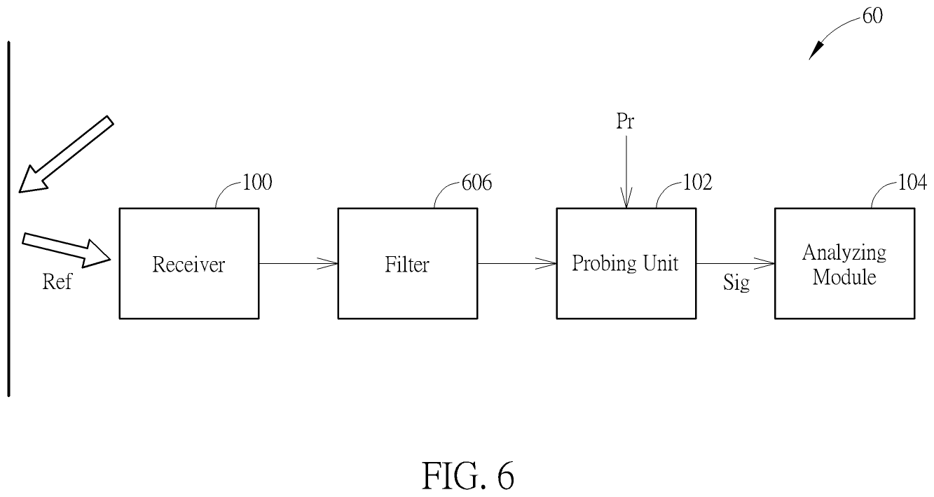

For example, please refer to FIG. 6, which is a schematic diagram of another analyzing device 60 for analyzing the psychological signal according to an embodiment of the present invention. The analyzing device 60 is similar to the analyzing device 10, and thus, the same components are denoted by the same symbols. The psychological signal analyzing device 60 further comprises a filter 606, which is coupled between the receiver 100 and the probing unit 102 and utilized for filtering out unnecessary frequency bands in the reflective signal Ref. For example, if the analyzing device 60 intends to analyze and determine a heartbeat frequency of the user, a filtering range of the filter 606 may be designed according to a range of the heartbeat frequency, to filter frequency bands except for the range of the heartbeat frequency. As such, the analyzing device 60 of the embodiment may further decrease computation complexity through the filter 606, and accurately determine the psychological frequency of the user without being affected by the environmental noise. Certainly, the filter 606 in such embodiment may be integrated with at least one of the probing unit 102 and the analyzing module 104 according to the above mentioned SoC method.

In addition, the present invention analyzes the reflective signal according to the subspace analysis, for obtaining the psychological frequency of the user. Therefore, in some embodiments, in addition to the MUSIC algorithm, the analyzing module may further analyze the reflective signal according to a singular value decomposition (SVD) method for determining the frequency of the psychological signal, as long as the analyzing module may determine the eigenvalues and the eigenvectors from the noise subspace and the signal subspace according to the subspace analysis. Moreover, the analyzing module may also analyze the reflective signal for determining the frequency of the psychological signal according to an eigenvalue decomposition (EVD) method.

Therefore, the analyzing module of the present invention may precisely analyze the frequency of the psychological signal without additional hardware. Notably, the analyzing device of the present invention is not limited to applications which obtain the psychological signal through a non-contact way, and may also be utilized for applications which obtain the psychological signal through a contact way, for analyzing the frequency of the psychological signal. For example, the analyzing device of the present invention may be a wearable device (e.g., a wristband, a watch, a finger sleeve, a smart suit, etc.) which may be worn on by the user for obtaining the psychological signal. Furthermore, the analyzing device of the present invention may also obtain the psychological signal of the user through a long/short term electrode patch.

In summary, the psychological signal obtained through the conventional non-contact way is lower in accuracy and reliability for determining the frequency of the psychological signal while the reflective signal is vulnerable to the noise interference. In comparison, the analyzing method and the analyzing device of the present invention may determine the frequency of the psychological signal from the reflective signal according to the subspace analysis, which may obtain the frequency of the psychological signal of the user fast and accurately. Moreover, under such a circumstance that no additional hardware is required, the analyzing method and the analyzing device of the present invention may be applied to applications which obtain the psychological signal through both the non-contact and the contact ways, further increasing hardware compatibility.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of analyzing a psychological signal, comprising:
receiving a reflective signal corresponding to the psychological signal reflected by a user;
performing summation to the reflective signal and a probe signal having a first frequency, to generate a summation signal; and
performing a subspace analysis of a first dimension to the summation signal, to generate an analytic result, to determine a frequency of the psychological signal.

2. The method of claim 1, further comprising:
estimating a power of the reflective signal, to determine a power of the probe signal.

3. The method of claim 1, further comprising:
filtering the reflective signal, to preserve a first frequency band which comprises the frequency of the psychological signal in the reflective signal.

4. The method of claim 1, wherein the step of performing the subspace analysis of the first dimension to the summation signal, to generate the analytic result comprises:
when the analytic result indicates that an intensity of the summation signal at the first frequency is greater than a predetermined intensity, determining the frequency of the psychological signal of the user.

5. The method of claim 4, wherein the step of performing the subspace analysis of the first dimension to the summation signal, to generate the analytic result comprises:
when the analytic result indicates that the intensity of the summation signal at the first frequency is smaller than or equal to the predetermined intensity, increasing the first dimension by a predetermined dimension, and repeatedly performing the subspace analysis until the analytic result indicates that the intensity of the summation signal at the first frequency is greater than the predetermined intensity.

6. The method of claim 1, wherein the step of performing summation to the reflective signal and the probe signal having the first frequency further comprises:
recording an amplitude of the reflective signal, so as to convert the reflective signal from a time domain to a frequency domain.

7. The method of claim 1, wherein the frequency of the psychological signal corresponds to at least one of a heartbeat, a breathe and a blood pressure of the user.

8. An analyzing device, for analyzing a psychological signal, the analyzing device comprising:
a receiver, configured to receive a reflective signal corresponding to the psychological signal reflected by a user;
a probing unit, configured to perform summation to the reflective signal and a probe signal having a first frequency, to generate a summation signal; and
an analyzing module, configured to perform a subspace analysis of a first dimension to the summation signal, to generate an analytic result, to determine a frequency of the psychological signal.

9. The analyzing device of claim 8, wherein the probing unit is further configured to perform the following step:
estimating a power of the reflective signal, to determine a power of the probe signal.

10. The analyzing device of claim 8, further comprising a filter, configured to perform the following step:
filtering the reflective signal, to preserve a first frequency band which comprises the frequency of the psychological signal in the reflective signal.

11. The analyzing device of claim 8, wherein the analyzing module is further configured to perform the following step, to perform the subspace analysis of the first dimension to the summation signal, to generate the analytic result:
when the analytic result indicates that an intensity of the summation signal at the first frequency is greater than a predetermined intensity, determining the frequency of the psychological signal of the user.

12. The analyzing device of claim 11, wherein the analyzing module is further configured to perform the following step, to perform the subspace analysis of the first dimension to the summation signal, to generate the analytic result:
when the analytic result indicates that the intensity of the summation signal at the first frequency is smaller than or equal to the predetermined intensity, increasing the first dimension by a predetermined dimension, and repeatedly performing the subspace analysis, until the analytic result indicates that the intensity of the summation signal at the first frequency is greater than the predetermined intensity.

13. The analyzing device of claim 8, wherein the probing unit is further configured to perform the following step, to perform summation to the reflective signal and the probe signal having the first frequency:
recording an amplitude of the reflective signal, so as to convert the reflective signal from a time domain to a frequency domain.

14. The analyzing device of claim 8, wherein the frequency of the psychological signal corresponds to at least one of a heartbeat, a breathe and a blood pressure of the user.
